SEQUENCE LISTING (18 sequences)

SEQ ID NO: 1 - DNA, 21 bp, Artificial Sequence
  DNA/RNA primer
ucuccgaggucuaaaguaunn

SEQ ID NO: 2 - DNA, 21 bp, Artificial Sequence
  DNA/RNA primer
auacuuuagaccucggagann

SEQ ID NO: 3 - DNA, 21 bp, Artificial Sequence
  DNA/RNA primer
gcacguucaauuccugguunn

SEQ ID NO: 4 - DNA, 21 bp, Artificial Sequence
  DNA/RNA primer
aaccaggaauugaacgugcnn

SEQ ID NO: 5 - DNA, 21 bp, Artificial Sequence
  DNA/RNA primer
ccuacagagaacugcgguunn

SEQ ID NO: 6 - DNA, 21 bp, Artificial Sequence
  DNA/RNA primer
aaccgcaguucucuguaggnn

SEQ ID NO: 7 - DNA, 21 bp, Artificial Sequence
  DNA/RNA primer
ggaccugagcagcaucuucnn

SEQ ID NO: 8 - DNA, 21 bp, Artificial Sequence
  DNA/RNA primer
gaagaugcugcucagguccnn

SEQ ID NO: 9 - DNA, 21 bp, Artificial Sequence
  DNA/RNA primer
guccaucauucaugcgaaann

SEQ ID NO: 10 - DNA, 21 bp, Artificial Sequence
  DNA/RNA primer
uuucgcaugaaugauggacnn

SEQ ID NO: 11 - DNA, 21 bp, Artificial Sequence
  DNA/RNA primer
ggagcucacuuaccaggaann

SEQ ID NO: 12 - DNA, 21 bp, Artificial Sequence
  DNA/RNA primer
uuccugguaagugagcuccnn

SEQ ID NO: 13 - DNA, 21 bp, Artificial Sequence
  DNA/RNA primer
gaagcucuccagaccauuunn

SEQ ID NO: 14 - DNA, 21 bp, Artificial Sequence
  DNA/RNA primer
aaauggucuggagagcuucnn

SEQ ID NO: 15 - DNA, 21 bp, Artificial Sequence
  DNA/RNA primer
gcgacuacauugaccagcunn

SEQ ID NO: 16 - DNA, 21 bp, Artificial Sequence
  DNA/RNA primer
agcuggucaauguagucgcnn

SEQ ID NO: 17 - protein, 361 aa, Homo sapiens
PRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQAGAHQGARLALDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKP

SEQ ID NO: 18 - protein, 351 aa, Homo sapiens
ERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLD

(19) United States
(12) Patent Application Publication
(10) Pub. No.: US 2005/0043212 A1
(43) Pub. Date: Feb. 24, 2005

(54) Differential inhibition of p38 map kinase isoforms

(76) Inventors: Ford Kirschenbaum, San Mateo, CA (US); Linda S. Higgins, Palo Alto, CA (US); George F. Schreiner, Los Altos, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/830,834

(22) Filed: Apr. 22, 2004

Related U.S. Application Data
(63) Continuation-in-part of application No. 10/683,656, filed on Oct. 9, 2003.
(60) Provisional application No. 60/417,599, filed on Oct. 9, 2002.

(51) Int. Cl. A61K 31/00; G06F 19/00; G01N 33/48; G01N 33/50

(52) U.S. Cl. 514/1; 702/19

(57) ABSTRACT

The invention is directed to methods and compositions that differentially inhibit the α-isoform of p38 MAP kinase.

### RELATED APPLICATIONS

This application is a continuation-in-part of U.S. patent application Ser. No. 10/683,656, which claims priority to U.S. Provisional Patent Application No. 60/417,599, filed Oct. 9, 2002, both of which are hereby incorporated by reference in their entirety.

### FIELD OF THE INVENTION

The invention relates to compounds useful in treating various disorders associated with enhanced activity of kinase p38. More specifically, it concerns compounds that differentially inhibit the α-isoform of p38 MAP kinase.

### BACKGROUND ART

The p38 MAP kinase family comprises a group of MAP kinases designated p38-α, p38-β, p38-γ and p38-δ. Jiang, Y., et al., J Biol Chem (1996) 271:17920-17926 reported characterization of p38-β as a 372-amino acid protein closely related to p38-α. In comparing the activity of p38-α with that of p38-β, the authors state that while both are activated by proinflammatory cytokines and environmental stress, p38-β was preferentially activated by MAP kinase kinase-6 (MKK6) and preferentially activated transcription factor 2, thus suggesting that separate mechanisms for action may be associated with these forms.

Kumar, S., et al., Biochem Biophys Res Comm (1997) 235:533-538 and Stein, B., et al., J Biol Chem (1997) 272:19509-19517 reported a second isoform of p38-β, -38-β2, containing 364 amino acids with 73% identity to p38-α. All of these reports show evidence that p38-β is activated by proinflammatory cytokines and environmental stress, although the second reported p38-β isoform, p38-β2, appears to be preferentially expressed in the central nervous system (CNS), heart and skeletal muscle, compared to the more ubiquitous tissue expression of p38-α. Furthermore, activated transcription factor-2 (ATF-2) was observed to be a better substrate for p38-β2 than for p38-α, thus suggesting that separate mechanisms of action may be associated with these forms. The physiological role of p38-β1 has been called into question by the latter two reports since it cannot be found in human tissue and does not exhibit appreciable kinase activity with the substrates of p38-α.

The identification of p38-γ was reported by Li, Z., et al., Biochem Biophys Res Comm (1996) 228:334-340 and of p38-δ by Wang, X., et al., J Biol Chem (1997) 272:23668-23674 and by Kumar, S., et al., Biochem Biophys Res Comm (1997) 235:533-538. The data suggest that these two p38 isoforms (γ and δ) represent a unique subset of the MAPK family based on their tissue expression patterns, substrate utilization, response to direct and indirect stimuli, and susceptibility to kinase inhibitors.

p38-α and β are closely related, but diverge from γ and δ, which are more closely related to each other. These protein kinases are activated by phosphorylation by upstream kinases MKK3, MKK6, and possibly by autophosphorylation. p38-α has been extensively studied and has been shown to play a causal role in inflammatory responses and in stress responses including apoptosis. p38-β has been less extensively studied. Nevertheless, these isoforms have distinct temporal and spatial patterns of expression, suggesting unique roles.

A large number of chronic and acute conditions have been recognized to be associated with perturbation of the inflammatory response. A large number of cytokines participate in this response, including IL-1, IL-6, IL-8 and TNF. It appears that the activity of these cytokines in the regulation of inflammation rely at least in part on the activation of an enzyme on the cell signaling pathway, a member of the MAP kinase family generally known as p38 and alternatively known as CSBP and RK. This kinase is activated by dual phosphorylation after stimulation by physiochemical stress, treatment with lipopolysaccharides or with proinflammatory cytokines such as IL-1 and TNF. Therefore, compounds that differentially inhibit the kinase activity of the α-isoform of p38 MAP kinase are useful agents.

### SUMMARY OF THE INVENTION

In one embodiment, the present invention is directed to the surprising and unexpected discovery that inhibition of the beta isoform of p38 MAP kinase results in what appears to be a compensatory upregulation in the levels and activity of p38 alpha. In another embodiment, the invention is directed to pharmacophore definition and three dimensional structure activity relationship for designing selective inhibitors of the alpha isoform of p38 MAP kinase. The invention is therefore directed to potent methods and compounds for antagonism of p38 alpha mediated disorders as well as potential new therapeutic targets within the compensatory mechanism.

### BRIEF DESCRIPTION OF THE DRAWINGS

FIG. 1A-1C shows an amino acid sequence alignment for the human α and β isoforms of p38 MAP kinase.

FIGS. 2A and 2B show ball and stick models of p38 MAP kinase inhibitors interacting with the alpha and beta isoforms of p38 MAP kinase.

FIGS. 3A and 3B show models of a preferred p38- MAP kinase inhibitor interacting with alpha and beta isoforms of p38 MAP kinase from two different orientations.

FIGS. 4A and 4B shows ball and stick models of p38 MAP kinase inhibitors interaction with the alpha and beta isoforms of p38 MAP kinase and regions of hydrophobicity on the inhibitor and the enzyme.

FIG. 5 shows a model of a SB203580 modified to improve p38 isoform selectivity.

FIG. 6 shows another model of a SB203580 modified to improve p38 isoform selectivity.

FIG. 7A shows a Western blot analysis of p38-α and actin expression levels produced from siRNA inhibition of p38-α kinase at 72 hours. FIG. 7B is a bar graph showing expression levels of p38-α kinase at 65 hours.

FIG. 8A shows a Western blot analysis of p38-α and actin expression levels produced from siRNA inhibition of p38-β kinase at 65 hours. FIG. 8B is a bar graph showing expression levels of p38-β kinase at 65 hours.

FIG. 9 is a bar graph showing levels of mRNA produced in human lung fibroblasts (HLFs) after 72 hours of treatment with siRNA to p38-α kinase (n=3).

FIG. 10 is a bar graph showing levels of mRNA produced in human lung fibroblasts (HLFs) after 72 hours of treatment with siRNA to p38-β kinase (n=3).

FIG. 11A-11D show bar graphs indicating the impact of p38-α and p38-β siRNAs on expression levels (18S RNA) of p38 isoforms α, β, and γ, and δ.

FIG. 12A shows a Western blot of HLF cells treated with 10 ng/ml of TNFα for 6 hours and siRNAs JA1 (p38-α), J4B (p38-β), A4 (p38-α), B11 (p38-α), 1AN (p38-α), J5B (p38-β), 4AN (p38-α), 1BN (p38-β), and a control oligo and the impact on phosphorylated Hsp 27 and phosphorylated p38-α levels. FIG. 12B is a bar graph indicating phosphorylated p38-α levels.

FIG. 13 is a Western blot analysis of siRNA treated (65 hours) HLF cells showing phosphorylated p38 (˜P p38) and phosphorylated heat shock protein 27 (˜P Hsp 27) in the presence “+” or absence “−“ of tumor necrosis factor-alpha (TNFα), which is known to induce p38 and Hsp 27 phosphorylation. The “Mock” column shows results from cells that were mock transfected. The “SCR” column shows results from cells into which a control siRNA was introduced. The “A4” column shows results from cells into which the siRNA construct A4 was introduced. The “B11” column shows results from cells into which the siRNA construct B11 was introduced.

FIG. 14 is a Western blot showing HLF cells treated with a p38-β targeted siRNA (B) increases phosphorylation of p38 while cells treated with a p38-α targeted siRNA (A) decreased phosphorylation of p38-α. The numbers at the top of each column indicate the time point in hours at which the sample was treated with TNFα. Cells were treated with siRNA for 65 hours.

FIG. 15 is a bar graph comparing levels of COX-2 18S RNA produced in HLF cells in response to treatment with OLIGOFECTAMINE (O), p38-α targeted siRNA, p38-β targeted siRNA, a control siRNA (Scramble), and O with a p38 inhibitor.

FIG. 16 is a bar graph comparing interleukin 1 (IL1) 18S RNA levels produced in HLFs cells (n=3) treated with a mock siRNA preparation, a control siRNA (SCR), a p38-α targeted siRNA (A4), or p38-β targeted siRNA (B11).

FIG. 17 is a Western blot showing the effect of siRNA treatment on HLF cells in the presence and absence of 10 ng TNFα for 17 hours. U=untreated cells; SCR=control siRNA; A=p38-α siRNA; B=p38-β siRNA; and ˜P Hsp 27=phosphorylated heat shock protein 27. The untreated cells (no siRNA) were exposed to 10 ng TNFα and to 10 ng TNFα+1.0 μM of a p38 inhibitor.

FIG. 18 is a plot of PGE2 inhibition by increasing concentrations of p38 MAP kinase inhibitor Compound 5.

### DETAILED DESCRIPTION OF THE PREFERRED EMBODIMENTS

### Detailed Description

### Inhibition of p38β MAP Kinase Isoform Induces Compensation

It has been discovered that the downregulation of p38β can lead to a compensatory upregulation of p38 alpha. This surprising effect can diminish the potency of nonselective p38 antagonists resulting in a loss of benefit. With respect to the therapeutic treatment of conditions mediated or characterized by enhanced p38-α MAP kinase activity, this observation may be the reason why despite the great deal of interest that has been expressed in developing inhibitors of p38 MAP kinase, little clinical success has resulted to date.

In one example of the compensatory capabilities of the p38 MAP kinase pathway, the p38-β MAP kinase isoform was downregulated using small interfering RNAs to prevent translation of beta-isoform mRNA. This inhibition of p38-β MAP kinase activity resulted in an increase both in p38-α isoform levels as well as an increase in phosphorylation of the downstream target of the p38 MAP kinase pathway, the heat shock protein Hsp27. The negative implications of the compensatory capabilities of the p38 MAP kinase pathway indicate that inhibitors of the p38 pathway are preferably isoform specific.

### Differences Between p38 α and β

The p38 Mitogen Activated Protein (MAP) kinase family of protein kinases comprises four isoforms: α, β, γ, and δ. These kinases are potently activated by a wide variety of stresses and cellular insults, such as inflammatory cytokines like tumor necrosis factor alpha (TNF-α) and interleukin-1 (IL-1). While p38α and β are closely related, they seem to diverge from the γ and δ isoforms.

P38α has been extensively studied and has been shown to play a causal role in inflammatory responses and in stress responses such as apoptosis. Comparatively, p38β has been less extensively studied. Differences in temporal and spatial patterns of expression suggest unique roles for the isoforms.

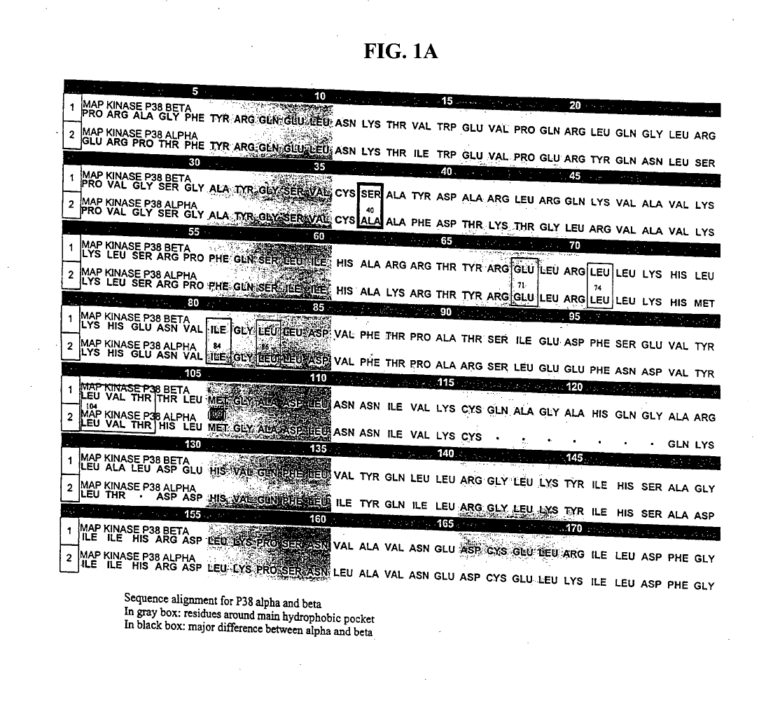

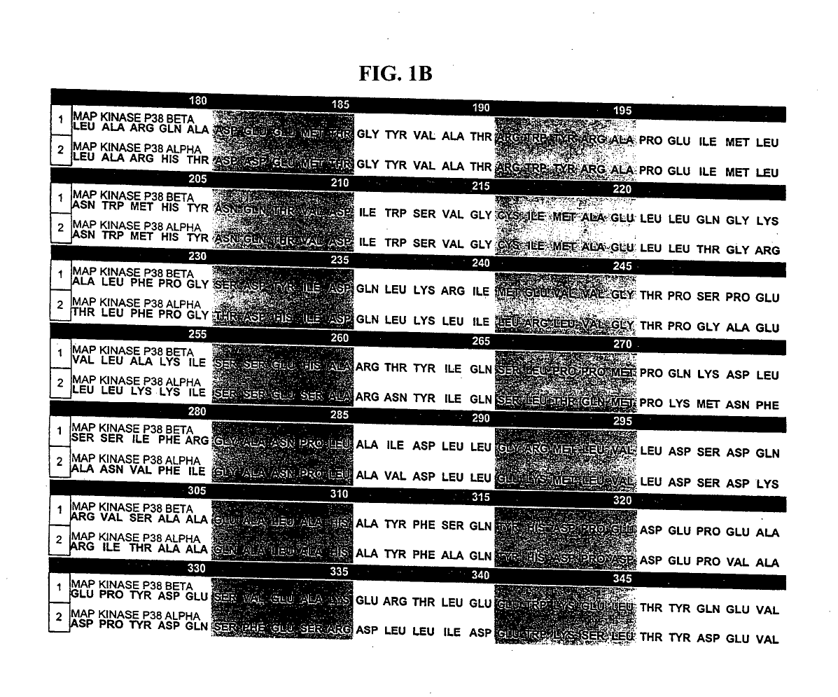

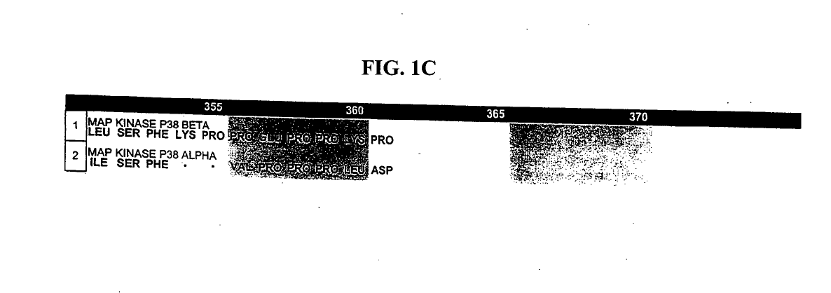

Based upon alignment and homology modeling studies, the relevant differences between the α and β isoforms are highlighted in FIG. 1A-C. In comparing sequences, there seem to be a number of differences between the isoforms. Taking into account alignment and homology however, one seemingly relevant difference is that the alpha isoform incorporates an alanine (ALA) residue at the position corresponding to position 40 in the figures while the beta isoform incorporates a corresponding serine (SER) residue. This is a difference which occurs within or in close proximity to the ATP binding pocket, a focal point for the enzymes' mechanism of action. It is thought that the presence of the non-polar ALA residue at this position with other residues proximate to the ALA create a small hydrophobic region that is unique to the alpha isoform.

### Pharmacophore Definition and Three-Dimensional Structure-Activity Relationship

The four isoforms of the p38 MAP kinase share a high level of sequence homology. The alpha and beta isoforms of the p38 MAP kinase are closely related while the gamma and delta isoforms are more divergent. Given the high degree of structural similarity, it is not surprising that certain compounds with the ability to inhibit one p38 MAP kinase isoform can often inhibit other isoforms of the MAP kinase. In a preferred embodiment, an inhibitor of p38 MAP kinase that is specific for the α-isoform of the kinase possesses at least three categories of structural features that are theorized to permit isoform specific inhibition.

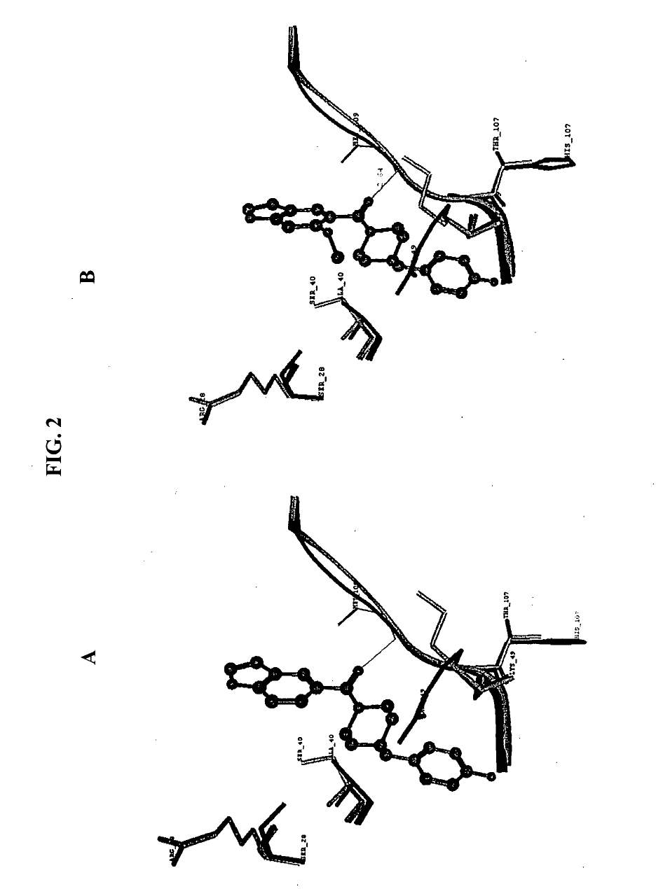

In binding to the enzyme, preferred embodiments of the p38 MAP kinase inhibitors preferentially bind at or near the ATP binding site of the kinases. A model of p38 MAP kinase inhibitors interacting with the alpha and beta isoforms of the p38 MAP kinase is shown in FIGS. 2-4. The models shown were generated using Molecular Operating Environment (MOE) software (version 2002.03) from Chemical Computing Group Inc. (Montreal, Quebec, CANADA). According to the manufacturer, MOE contains an integrated force field engine with open parameterization. Parameters for AMBER '89/'94, CHARMM22, MMFF94, MMFF94s, OPLS-AA and Engh-Huber models are included with the distribution. Three large-scale energy minimization algorithms are available, and distance, angle and torsion angle constraints can be imposed. All force fields include an implicit solvent model energy term.

As shown in representations of the model, there are at least three regions of interact between a preferred embodiment of a model inhibitor (shown below) and the α-isoform of the p38 MAP kinase.

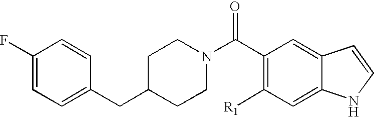

Discussed in no particular order, the first structural feature suggested as being important by molecular modeling relates to an amino acid difference existing between the human p38-α and p38-β isoforms of the MAP kinase. As shown in FIG. 1, the black boxed residue of the figure indicates an amino acid difference in a putative specificity-determining pocket of the kinase. As mentioned above, the alpha and beta isoforms of the p38 MAP kinase differ at position 40, where the alpha isoform has an alanine residue (ALA) and the beta isoform has a serine residue (SER). It is hypothesized that the presence of the non-polar ALA residue at this position with the other residues proximate to the ALA creates a hydrophobic region on this portion of the kinase. Gray boxed residues at 71, 74, 84, 86, and 104-106 indicate residues located in the adenosine triphosphate (ATP) binding pocket of the kinase which further define an addition and more major hydrophobic region. Accordingly, the first position of interest on the model inhibitor relates to substituents at position R₁ of the model compound.

As suggested by the model, a hydrophobic or non-polar group positioned at R₁ allows for preferred embodiments of the disclosed inhibitors to interact with the α-isoform of p38 MAP kinase in an isoform specific manner. Examples of preferred substituents at this position include hydrocarbyl residues (1-6C) containing 0-2 heteroatoms selected from O, S and/or N and inorganic residues. Preferably, the substituents represented by R₁ are independently halo, alkyl, heteroalkyl, or NR₂, wherein R is H, alkyl, aryl, or heteroforms thereof. More preferably R¹ substituents are selected from alkyl, alkoxy or halo, and most preferably methoxy, methyl, and chloro. Most preferably, R¹ is halo, lower alkyl, methoxy or ethoxy.

The second feature suggested as important to designing isoform specific inhibitors of the p38 MAP kinase concerns a hydrogen bond donor in the “hinge region” of the enzyme. Docking of the lowest energy conformations of preferred embodiments of the disclosed inhibitors into the ATP site of p38α suggests that the amide carbonyl of the model compound forms an important hydrogen bond with the backbone amide proton of the methionine residue at position 109 in the hinge region of p38α. Accordingly, in a preferred embodiment, an α-isoform specific p38 MAP kinase inhibitor will possess a hydrogen bond acceptor moiety that interacts with the ATP binding site at or near the position occupied by MET 109.

The importance of the MET109 residue in binding inhibitors is illustrated by the work of Eyers, et al., “Conversion of SB 203590-insentisive MAP kinase family members to drug-sensitive forms by a single amino-acid substitution,” Chem Biol. (1998) 5(6):321-328. This study demonstrates that the MET109 of p38-α conveyed isoform-specific functional characteristics with respect to inhibitor sensitivity that were altered by site-directed mutagenesis of this residue.

Another important feature of the preferred α-isoform specific p38 MAP kinase inhibitors concerns interaction with a second and more prominent hydrophobic pocket in both p38 alpha and beta isoforms. A hydrophobic interaction with a hydrophobic moiety such as an aryl group is exemplified by the model above as a substituted benzyl. From previous studies it was shown that this aryl group can play a role in providing basic activity to the described class of inhibitors, albeit not necessarily in an isoform-specific manner. The aryl at this position may be a heteroaryl, including 6-5 fused heteroaryl, cycloaliphatic or cycloheteroaliphatic that can be optionally substituted. The aryl group is preferably optionally substituted phenyl.

Each substituent on the aryl group can independently be a hydrocarbyl residue (1-20C) containing 0-5 heteroatoms selected from O, S and N, or is an inorganic residue. Preferred substituents include those selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two of said optional substituents on adjacent positions can be joined to form a fused, optionally substituted aromatic or nonaromatic, saturated or unsaturated ring which contains 3-8 members. More preferred substituents include halo, alkyl (1-4C) and more preferably, fluoro, chloro and methyl. These substituents may occupy all available positions of the aryl ring, preferably 1-2 positions, most preferably one position. These substituents may be optionally substituted with substituents similar to those listed. Of course some substituents, such as halo, are not further substituted, as known to one skilled in the art. Two substituents on the aryl ring can be joined to form a fused, optionally substituted aromatic or nonaromatic, saturated or unsaturated ring which contains 3-8 members.

The aryl ring at this position is thought to be important for inhibitory activity. Modeling of a preferred inhibitor suggests an enzyme interaction with the pictured benzyl group that occupies a hydrophobic pocket adjacent to the ATP site of the enzyme (FIGS. 4A and 4B).

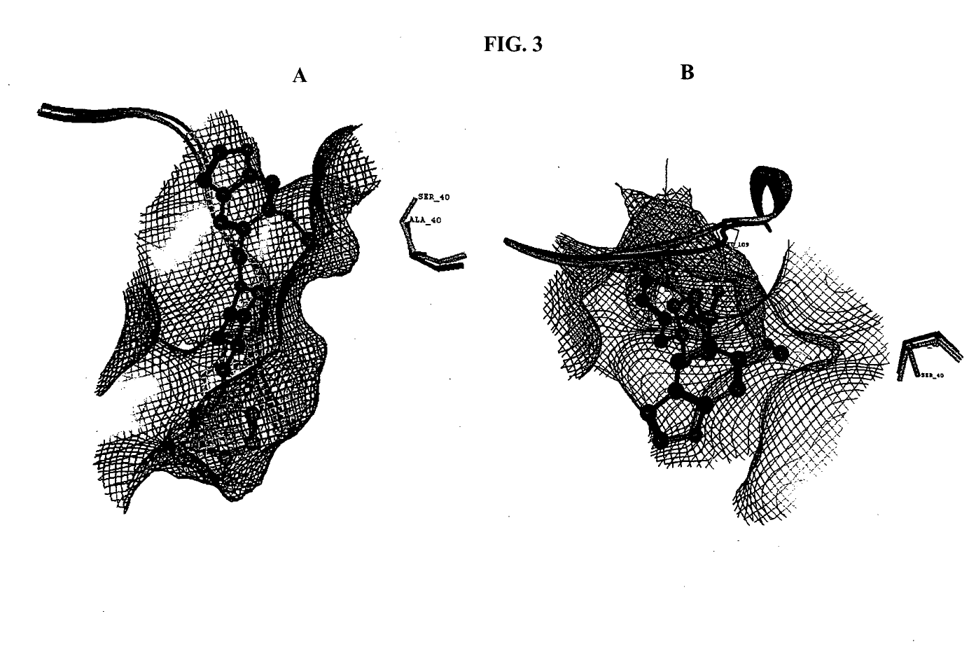

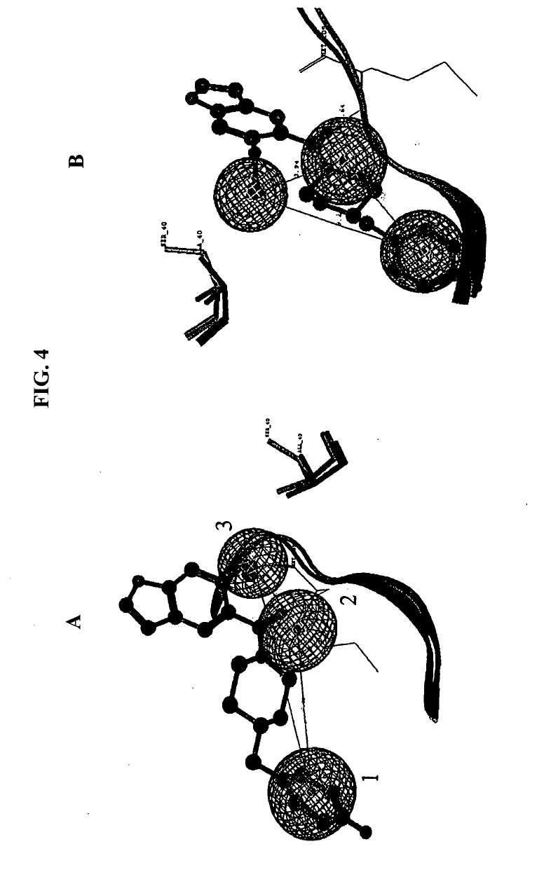

FIG. 3 shows a preferred p38α isoform specific MAP kinase inhibitor from two vantage points. The model shows the amide carbonyl interacting with MET109 and shows the location of a hydrophobic moiety at R₁ ortho to the carbonyl group interacting with a hydrophobic pocket of the kinase. The model also indicates the interaction of the indole and the benzyl group relative to the kinase.

In a non-limiting theory of the invention, it is hypothesized that the structural features of the preferred inhibitors provide the criteria necessary to design and identify potent inhibitors of the p38 MAP kinase that are specific or at least bind with differential specificity to the alpha isoform of the kinase. These theorized components of a preferred p38-α MAP kinase inhibitor include a substituted or unsubstituted indole, benzimidazole, or other similar moiety and a substituted or unsubstituted piperidine or piperazine amide linked by the carbonyl group of the amide. In theory, as discussed above, the amide carbonyl forms a hydrogen bond with the methionine at position 109 of the kinase, which positions the inhibitor in an orientation to block the binding of ATP to the kinase. The substituted or unsubstituted piperidine or piperazine amide is hypothesized to interact with the kinase via hydrophobic or non-polar forces, which provide another anchor point with which to orient the inhibitor to the kinase. The specific nature of the structural elements of the invention's inhibitors is less important that the ability of these features to correctly orient the inhibitor with respect to the ATP binding site of the kinase. In addition, it is preferred that the elements of the model molecule as a whole contribute to a rigid backbone, necessary to maintain the requisite interaction with p38 alpha thereby supporting both activity and selectivity.

The general features discussed above are thought to provide important structural features to preferred p38 kinase inhibitors. It is hypothesized that substituents located ortho to the carbonyl moiety, taken in combination with the other features of the inhibitor, provide a combination of structural features that lend a degree of rigidity to the preferred inhibitors. In this non-limiting theory of the invention, a hydrophobic R₁ group limits the ability of the indole benzimidazole, or other similar moiety and the piperazine or piperidine groups to rotate about the bonds connecting these groups the central carbonyl group. This limited freedom of rotation may serve to hold the various features of the preferred inhibitors in a spatial configuration that allows the inhibitor to be both highly competitive with ATP for the ATP binding site and to maintain the position of the features of the inhibitor that make the preferred compounds more specific for the α-isoform of the p38 MAP kinase.

As shown in FIGS. 4A and 4B, distances among the three structural features of a preferred p38-isoform selective inhibitor are indicated by dots positioned in the middle of each of three balls indicating hydrophobicity. The calculated distance between the aryl hydrophobic group and the hydrogen bond acceptor group of the preferred inhibitors is approximately 6.8±3 Å. The calculated distance between the aryl group and the hydrophobic substituent of R₁ is approximately 9.2±2.5 Å. The calculated distance between the hydrogen bond acceptor and the hydrophobic substituent of R₁ is approximately 3.9±2.5 Å. These three points and the distances among them define a plane, a structural characteristic of the preferred embodiments of the disclosed invention.

### Designing Isoform Selective p38 MAP Kinase Inhibitors

From the structural and functional principles outlined above, it is possible for one of ordinary skill in the art to design novel isoform-selective inhibitors of the p38 MAP kinase. In short, an isoform specific inhibitor can be designed using any compound that binds to the ATP binding site of the kinase.

An example of such an exercise could use the nonselective p38 MAP kinase inhibitor SB203580 from CalBiochem (San Diego, Calif.). The structure of SB203580 is: 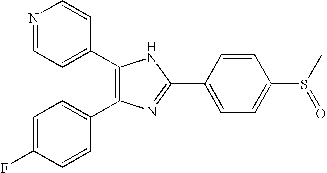
4-(4-Fluorophenyl)-2-(4-methylsulfinylphenyl)-5-(4-pyridyl) 1H-imidazole

Modification of this compound with the addition of a dicholorobenzyl group to the pyridine of SB203580 yields 2-(2,6-Dichloro-phenyl)-4-[5-(4-fluoro-phenyl)-2-(4-methanesulfinyl-phenyl)-3H-imidazol-4-yl]-pyridine, having the structure:

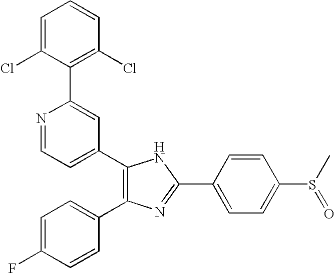

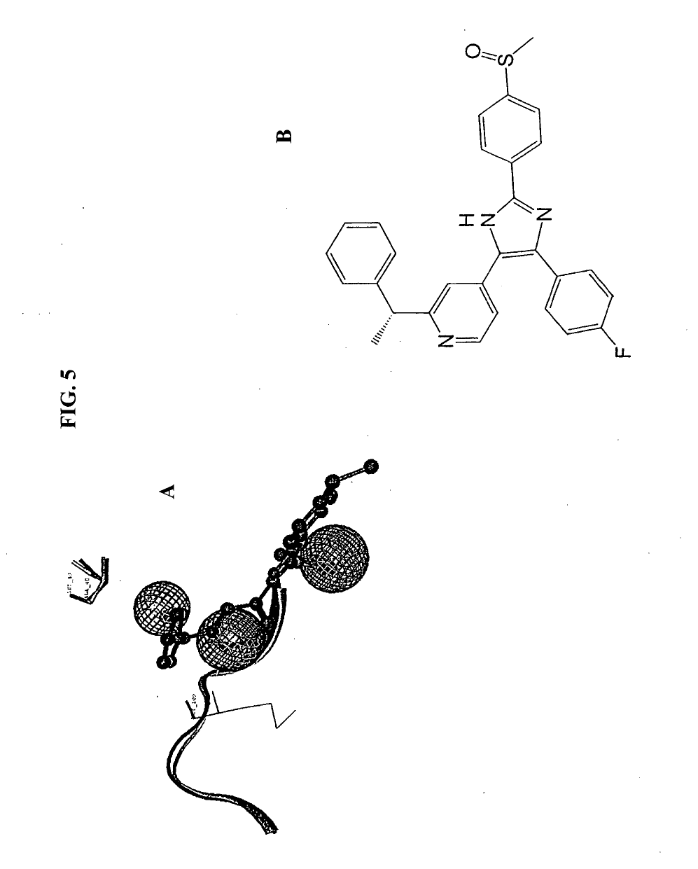

This modified compound was model as discussed above with the α-isoform of the kinase. As shown in FIG. 5, the addition of the dicholorobenzyl group provides a hydrophobic moiety with which the modified inhibitor can use to interact with ALA40 of the α-isoform. The addition of this group will thus make this isoform non-specific p38 MAP kinase inhibitor more selective for the α-isoform than the unmodified compound.

Another version of SB203580 is modified to increase the specificity of the inhibitor in accordance with the teachings of the disclosed invention. In this modified version, the SB203580 has been modified by adding an isopropyl-benzyl group to the pyridine of SB203580 to yield 4-[5-(4-Fluoro-phenyl)-2-(4-methanesulfinyl-phenyl)-3H-imidazol-4-yl]-2-(1-phenyl-ethyl)-pyridine which has a structure of:

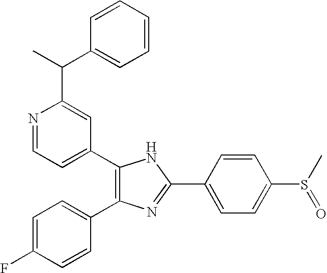

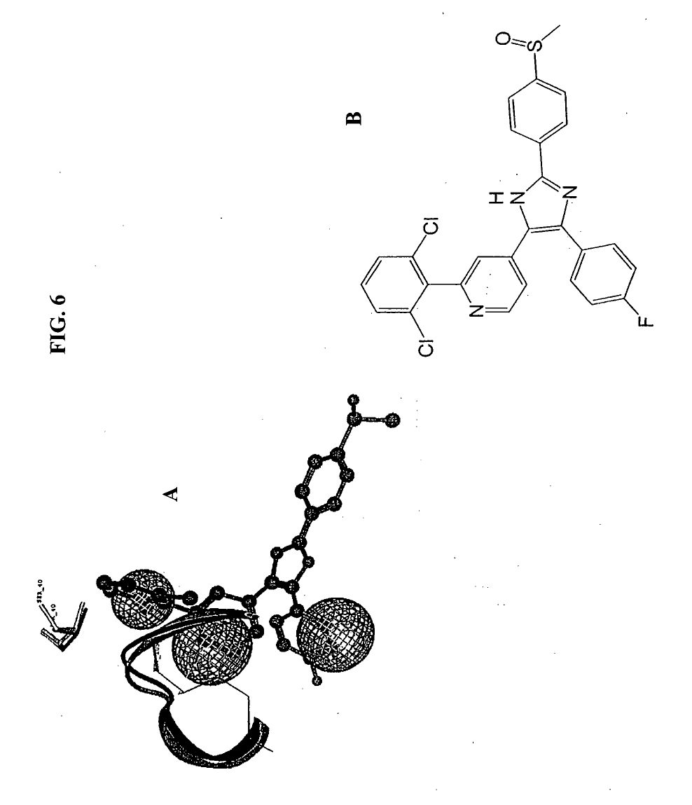

As shown in FIG. 6, the addition of the isopropyl-benzyl group provides a hydrophobic moiety with which the modified inhibitor uses to specifically interact with the α-isoform of p38 MAP kinase.

### Inhibitors of p38 MAP Kinase

The described invention also relates to the observation that inhibitors of p38 MAP kinase that are specific or to a degree selective for the α-isoform as opposed to other isoforms of the kinase. Preferably, an inhibitor of p38 MAP kinase will be 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500-fold more selective for the α-isoform than for other isoforms of the p38 MAP kinase.

Selectivity for the p38 MAP kinase inhibitor is a desirable quality. In experiments disclosed below, it has been discovered that inhibition of isoforms of the p38 MAP kinase other than the α-isoform of the enzyme results in the upregulation or compensation of other elements of the p38 MAP kinase cascade. This compensation effect results in the phosphorylation of downstream targets of the p38 cascade, which implies that the p38 MAP kinase cascade maintains its ability to induce inflammation and other undesirable effects when p38 MAP kinase isoforms other than the α-isoform of the enzyme are inhibited. In view of these results discussed below, the presently disclosed invention considers the identification and use of p38 MAP kinase inhibitors specific or selective for the α-isoform to be preferred.

Selective binding of a candidate p38 MAP kinase inhibitor can be determined by a variety of methods. The genes for the various isoforms of p38 MAP kinase are known in the art. One of ordinary skill in the art could readily clone and express the various isoforms of the kinase, purify them, and then perform binding studies with candidate compounds to determine isoform binding characteristics. This series of experiments was performed for the α-isoform of p38 MAP kinase and provided in U.S. Pat. No. 6,617,324 B1, which is hereby incorporated by reference in its entirety.

As used herein, the term “inhibitor” includes any suitable molecule, compound, formulation or substance that may regulate p38 MAP kinase activity, preferably the α-isoform of the kinase. The inhibitor may be a protein or fragment thereof, a small molecule compound, or even a nucleic acid molecule. In a preferred embodiment of the invention, the inhibitor regulates the α-isoform of p38 MAP kinase with at least 75 fold specificity for the α-isoform over the other isoforms of the kinase.

According to the present invention, the inhibitor may exhibit its regulatory effect upstream or downstream of p38 MAP kinase or on p38 MAP kinase directly. Examples of inhibitor regulated p38 MAP kinase activity include those where the inhibitor may decrease transcription and/or translation of p38 MAP kinase, may decrease or inhibit post-translational modification and/or cellular trafficking of p38 MAP kinase, or may shorten the half-life of p38 MAP kinase. The inhibitor may also reversibly or irreversibly bind p38 MAP kinase, inhibit its activation, inactivate its enzymatic activity, or otherwise interfere with its interaction with downstream substrates.

If acting on p38 MAP kinase directly, the inhibitor should exhibit an IC₅₀ value of about 5 μM or less, preferably 500 nm or less, more preferably 100 nm or less. In a related embodiment, the inhibitor should exhibit an IC₅₀ value relative to the p38-α isoform that is preferably at least ten fold less than that observed when the same inhibitor is tested against other p38 MAP kinase isoforms in the same or comparable assay. It should be noted that IC₅₀ values are assay dependent and may change from determination to determination. It is more important to look at relative relationships of compounds' IC₅₀ values rather than the exact values themselves.

### Assays for Determining p38 Kinase Inhibition

The following assays can be used to determine relative IC₅₀ values for p38 inhibitors. IC₅₀ is the relative concentration of an inhibitor, which in the presence of the target kinase, causes a 50% decrease in kinase activity as compared to a control where the inhibitor is not present.

In an exemplary assay, compounds to be tested are solubilized in DMSO and diluted into water to the desired concentrations. The p38 kinase is diluted to 10 μg/ml into a buffer containing 20 mM MOPS, pH 7.0, 25 mM beta-glycerol phosphate, 2 mg/ml gelatin, 0.5 mM EGTA, and 4 mM DTT.

A reaction is carried out by mixing 20 μl test compound with 10 μl of a substrate cocktail containing 500 μg/ml peptide substrate and 0.2 mM ATP (+200 μCi/ml gamma-32P-ATP) in a 4× assay buffer. The reaction is initiated by the addition of 10 μl of p38 kinase. Final assay conditions are 25 mM MOPS, pH 7.0, 26.25 mM beta-glycerol phosphate, 80 mM KCI, 22 mM MgCl₂, 3 mM MgSO₄, 1 mg/ml gelatin, 0.625 mM EGTA, 1 mM DTT, 125 μg/ml peptide substrate, 50 μM ATP, and 2.5 μg/ml enzyme. After a 40 minute incubation at room temperature, the reaction is stopped by the addition of 10 μl per reaction of 0.25 M phosphoric acid.

A portion of the reaction is spotted onto a disk of P81 phosphocellulose paper, the filters are dried for 2 minutes and then washed 4× in 75 mM H₃PO₄. The filters are rinsed briefly in 95% ethanol, dried, then placed in scintillation vials with liquid scintillation cocktail.

Alternatively, the substrate, previously biotinylated, and the resulting reactions are spotted on SAM²™ streptavidin filter squares (Promega). The filters are washed 4× in 2M NaCl, 4× in 2M NaCl with 1% phosphoric acid, 2× in water, and briefly in 95% ethanol. The filter squares are dried and placed in scintillation vials with liquid scintillation cocktail.

Counts incorporated are determined on a scintillation counter. Relative enzyme activity is calculated by subtracting background counts (counts measured in the absence of enzyme) from each result, and comparing the resulting counts to those obtained in the absence of inhibitor. IC₅₀ values are determined with curve-fitting plots available with common software packages.

In an alternative assay for measuring in vitro p38 kinase activity, human recombinant p38 (in this case either alpha or beta isoform) is mixed with the inhibitor at the desired concentration, and with DMSO kept at 1% in the final reaction. Myelin basic protein (MBP) and ATP are added and the reaction is incubated for 60 min at 25° C. The incubation conditions include 10 ug/ml MBP, 10 uM ATP, 50 mM HEPES, 20 mM MgCl₂, 0.2 mM Na VO₄, and 1 mM DTT, at pH 7.4. The degree of phosphorylation of the MBP is determined by ELISA quantitation of phospho-MBP.

As referenced above, IC₅₀ is the concentration of compound which inhibits the enzyme to 50% of the activity as measured in the absence of an inhibitor.

IC₅₀ values are calculated using the concentration of inhibitor that causes a 50% decrease as compared to a control. IC₅₀ values are assay dependent and will vary from measurement to measurement. As such, IC₅₀ values are relative values. The values assigned to a particular inhibitor are to be compared generally rather than on an absolute basis.

### Exemplary Inhibitors

The following table of compounds represents examples of α-isoform selective compounds.

| Compound | | alpha | beta | |
|---|---|---|---|---|
| Number | STRUCTURE | (nM) | (nM) | selectivity |
| 1 | 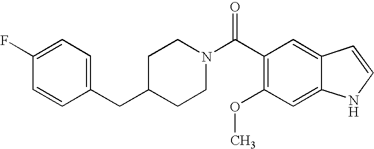 | | | |
| 2 | 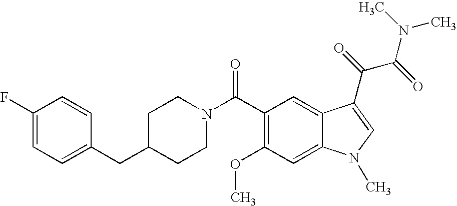 | 6.5 | 130 | 20 |
| 3 | 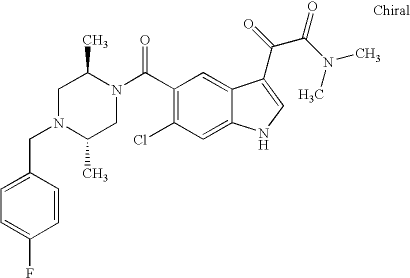 | | | |
| 4 | 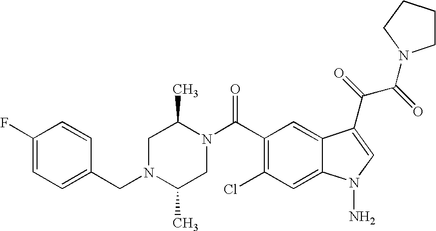 | 1.6 | 23 | 14.375 |
| 5 | 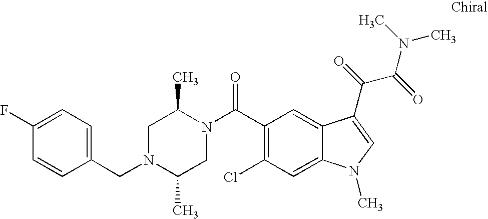 | 1.7 | 91 | 53.52941 |
| 6 | 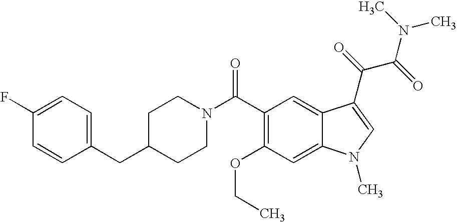 | 9 | 98 | 10.88889 |
| 7 | 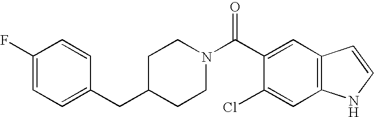 | | | |
| 8 | 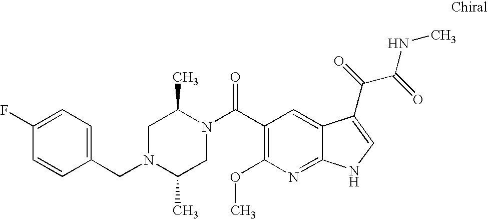 | 30 | 3020 | 100.6667 |
| 9 | 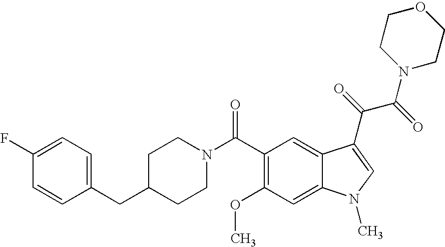 | 1.8 | 407 | 226.1111 |
| 10 | 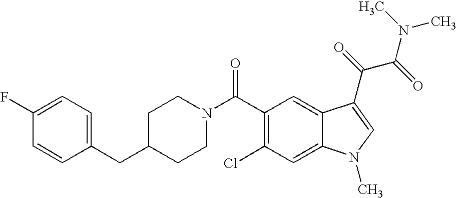 | 9 | 90 | 10 |
| 11 | 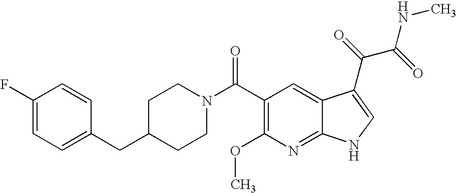 | 19 | 321 | 16.89474 |
| 12 | 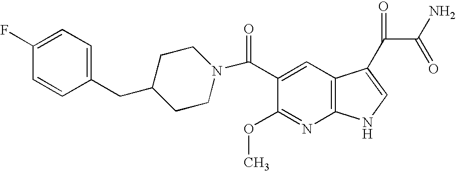 | 6.1 | 1520 | 249.1803 |

Within the scope of the invention are also pharmaceutically acceptable salts of the compounds of the P38 Kinase inhibitors.

Suitable pharmaceutically acceptable salts of the compounds of the p38 kinase inhibitors include the hydrochloride salt, the acetate salt and the trifluoroacetate salt.

The manner of administration and formulation of the compounds useful in the invention and their related compounds will depend on the nature of the condition, the severity of the condition, the particular subject to be treated, and the judgment of the practitioner; formulation will depend on mode of administration. As the compounds of the invention are small molecules, they are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

It should be noted that the compounds of the described invention can be administered as individual active ingredients, or as mixtures of several embodiments of this formula. In addition, the inhibitors of p38 kinase can be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation or activation.

As implied above, although the compounds of the invention may be used in humans, they are also available for veterinary use in treating animal subjects.

The following examples are intended to illustrate but not to limit the invention.

### EXAMPLE 1

### Inhibition of p38 β-Isoform by siRNA Shows Compensation by the p38 α-Isoform

RNA interference (RNAi) is a powerful method for analyzing gene function. RNAi is a post-transcriptional process where the introduction of double-stranded RNA (dsRNA) inhibits protein production in a sequence-specific manner. In vitro, RNAi is induced using double stranded segments of RNA (dsRNA) which are cleaved into short fragments of small interfering RNAs (siRNAs). These siRNAs form a ribonuclease RNA Induced Silencing Complex (RISC) which mediates the cleavage of a target mRNA.

Design of the siRNAs included providing an antisense strand of each siRNA which was targeted against an AA(N) 9 sequence in the target coding region of the gene of interest where the GC content was between 47-53%. Two online programs were used to identify potential siRNAs: “siRNA Design” (QIAGEN, Valencia, Calif.), which identified siRNAs that follow general design guidelines and “Mfold,” which predicted secondary RNA structure accessibility (Stewart, D. & Zuker, M., Washington University). siRNA sequences were subjected to BLAST analysis to preclude selection of sequences that showed significant similarity to other p38 isoforms as well as other genes in the human genome. siRNAs were obtained from DHARMACON (Boulder, Colo.) as annealed and purified duplexes. Sequences included (sense strand): MAPK14 (sequence homologous to all p38α splice variants: CSBP2, CSBP1, Mxi2, Exip) 5′-CCUACAGAGAACUGCGGUU-dTdT-3′ (SEQ ID NO: 5); MAPK11 (p38β) 5′-GGACCUGAGCAGCAUCUUC-dTdT-3′ (SEQ ID NO: 7).

Primary normal human lung fibroblasts (HLF) cells (CAMBREX, Baltimore, Md.) were seeded between 40-60% confluency in 6-well plates using fibroblast growth media (FGM) supplemented with 2% FBS, 5 μg/mL insulin and 1 ng/ml hFGF-B (CAMBREX) and grown at 37° (passage 9). Transfections for targeting endogenous genes were carried out the following day. Briefly, 30 μl of a 20 nM siRNA solution and 30 μl of OLIGOFECAMINE (INVITROGEN, Carlsbad, Calif.) were complexed for 20 minutes in 1,140 μl of OPTI-MEM I (INVITROGEN) to facilitate complex formation. 200 μl of the resulting mixture was added to each well of cells culture in a 6-well plate containing 800 μl of serum free FBS media. For Western blots, cells were lysed in RIPA buffer supplemented with protease inhibitors. Lysates were quantified by BCA assay and equal amounts of protein were loaded and electrophoresed on SDS-PAGE gels. The proteins were blotted onto an appropriate membrane and antibodies against p38-α, p38-β and Hsp27 were used according to the manufacturer's recommendations.

Initial experiments demonstrated the ability of p38 MAP kinase isoform specific siRNA constructs to inhibit protein production in human lung fibroblasts. FIG. 1A shows a Western blot of human lung fibroblasts (HLFs) that were transfected with siRNA constructs, SCR (control), or p38-α-directed siRNAs A4, A7 and POOL (A4+A7). Actin levels were indicated as a house keeping control to indicate background expression levels. All three p38-α-directed siRNA-treated lanes indicated that p38-α expression was reduced. FIG. 1B graphs the data from the Western blot, which supports the observation that the p38-α-directed siRNAs were effective to reduce p38-α MAP kinase levels. FIG. 2A shows a Western blot of HLFs treated with a control siRNA (SCR), a p38-α-directed siRNA (A4) and a p38-β-directed siRNA (B11) on p38-β and heat shock protein 27 (Hsp27) expression levels. The p38-α-directed siRNA A4 had a minimal effect on p38-β and Hsp27 expression levels while the p38-β-directed siRNA B11 reduced both p38-β and Hsp27 expression levels. FIGS. 3 and 4 indicate that RNA from the alpha and beta isoforms of p38 was reduced by the presence of the p38-α- and β-directed siRNA. These results indicate that the siRNA constructs were functioning as intended.

The effects of different p38-α- and β-directed siRNAs on the RNA levels of the p38-α (FIG. 5A), p38-β (FIG. 5B), p38-γ (FIG. 5C), and p38-δ (FIG. 5D) isoforms was examined. In FIG. 5A, p38-α-directed siRNAs A4 and A7 reduced p38-α-RNA levels relative to the untreated control cells (U). Also, p38-α-directed siRNA B11 reduced p38-β-RNA levels relative to the untreated control cells (FIG. 5B). Interestingly, the p38-α-directed siRNAs A4 and A7 appeared to inhibit p38-γ-RNA production (FIG. 5C) while both the p38-α-directed siRNAs A4 and A7 and the p38-β-directed siRNA B11 elevated p38-δ-RNA levels, relative to the untreated control cells (FIG. 5D).

An experiment was performed looking at siRNAs directed to different unique sequences within the p38-α and p38-β isoform messages. Cells were transfected with siRNA constructs and exposed to TNF-α, an inflammatory cytokine and upstream inducer of the p38 MAP kinase pathway. As expected, cells transfected with constructs producing p38-α-directed siRNAs (JA1, A4, 1AN and 4AN) showed reduced levels of phosphorylated p38-α and Hsp27 as compared to the control lane labeled “Oligo” (FIG. 6A). Phosphorylated p38-α and Hsp27 was shown to be elevated in cells treated with p38-β-directed siRNAs (J4B, B11, J5B and 1BN). The results from the phosphorylated p38 MAP kinase were also plotted in bar graph form to more clearly indicate the inhibitory and stimulatory effects of the p38-α and p38-β-directed siRNAs (FIG. 6B). These results suggest that inhibition of p38-β-kinase isoform may stimulate rather than inhibit downstream events in the p38 MAP kinase pathway.

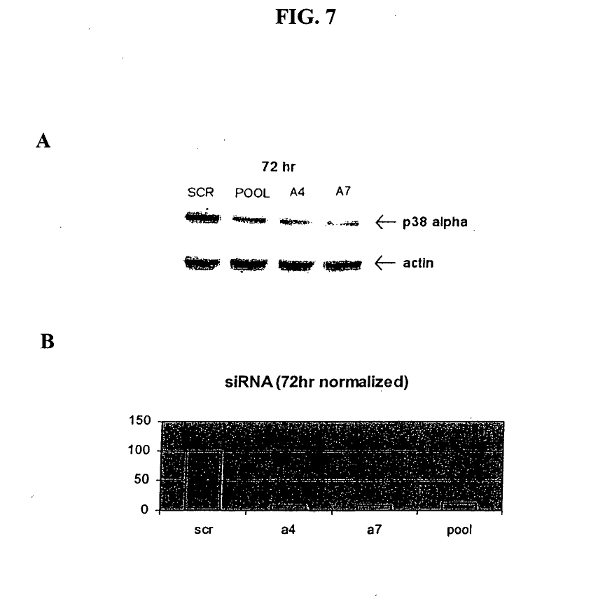

The downstream effects of p38-α- and β-directed siRNAs was examined in the next set of experiments. Western blots were made of HLF cells to examine the effects of the siRNAs on p38 and Hsp27 phosphorylation. Cells were exposed to TNF-α, a so-called “inflammatory cytokine,” which causes phosphorylation of p38 and is an upstream activator of the p38 MAP kinase pathway. Phosphorylation of Hsp27 occurs as a downstream event in the pathway. As shown in FIG. 7, the control cells (Mock and SCR) show little phosphorylated p38 or Hsp27 in the absence of TNF-α and a marked increase in phosphorylation in the presence of TNF-α. In cells treated with the p38-α-directed siRNA A4 showed a decrease in p38 phosphorylation and little effect on the levels of phosphorylated Hsp27 as compared to the control lanes. However, cells treated with the p38-β-directed siRNA B11 showed increases in both p38 and Hsp27 phosphorylation. These results suggest that inhibition of p38-β MAP kinase actually leads to an increase in downstream events of the p38 MAP kinase pathway. In other words, inhibition of p38β-kinase may actually activate the inflammation response pathway.

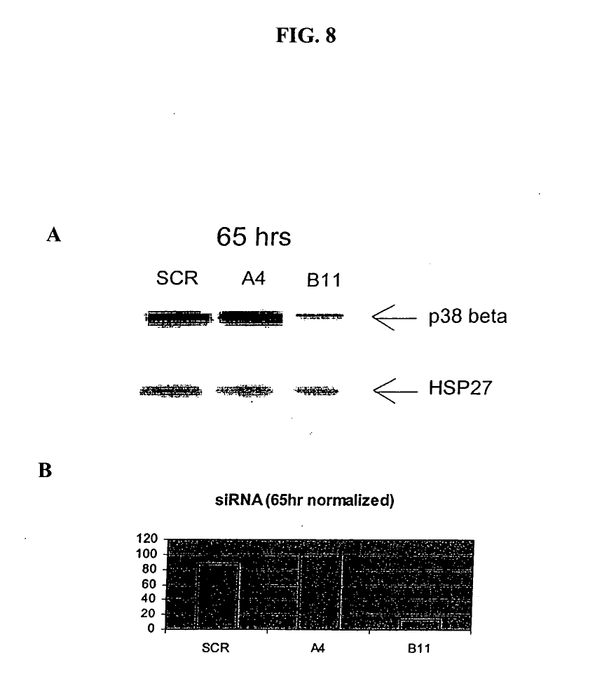

The effect of p38-β MAP kinase inhibition on downstream events of the p38 MAP kinase pathway was further examined with respect to p38-β MAP kinase expression levels, p38 phosphorylation, and Hsp27 levels. FIG. 8 shows a Western of HLF cells treated with a control siRNA (SCR), p38-α-directed siRNA (A) or a p38-β-directed siRNA (B) in the presence and absence of TNF-α. At each of the 3, 6, 9, and 18 hour time points, cells treated with the p38-α-directed siRNA showed reduced levels of phosphorylated p38, which is expected for an inhibitor of p38-α-kinase, with levels of p38-β-kinase being comparable to those of the control cells. In contrast, cells treated with the p38-β-directed siRNA showed the expected decrease in p38-β-kinase levels at all the time points, but also showed a marked increase in p38 phosphorylation as compared to the control lanes. These results support the conclusion that inhibition of p38β-kinase actually leads to an increase in downstream events in the p38 MAP kinase pathway.

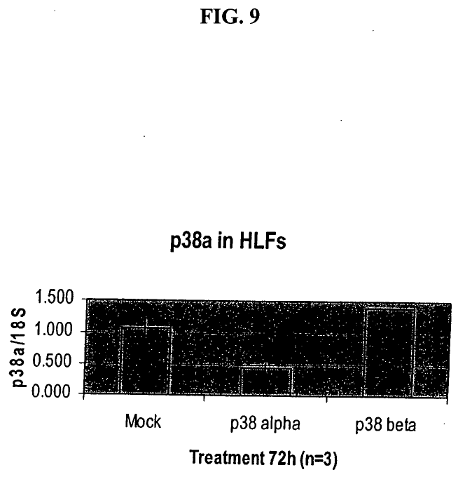

Cyclooxygenase-2 (Cox-2) is thought to play a role in inflammation and to be upregulated by the p38 MAP kinase pathway. HLF cells were transfected with various siRNA constructs or treated with a p38 MAP kinase inhibitor that demonstrated approximately a 10-fold specificity for the alpha isoform over the beta isoform. The impact of these conditions on Cox-2 RNA was then examined. As shown in FIG. 9, cells treated with the p38-β-directed siRNA showed a marked increase in Cox-2 RNA. In contrast, cells treated with the p38-α-directed siRNA, various controls, or the p38 MAP kinase inhibitor, all showed Cox-2 RNA levels similar to that of the untreated cells. These results show that inhibition of p38-β-kinase result in an upregulation of the inflammation-response associate enzyme Cox-2.

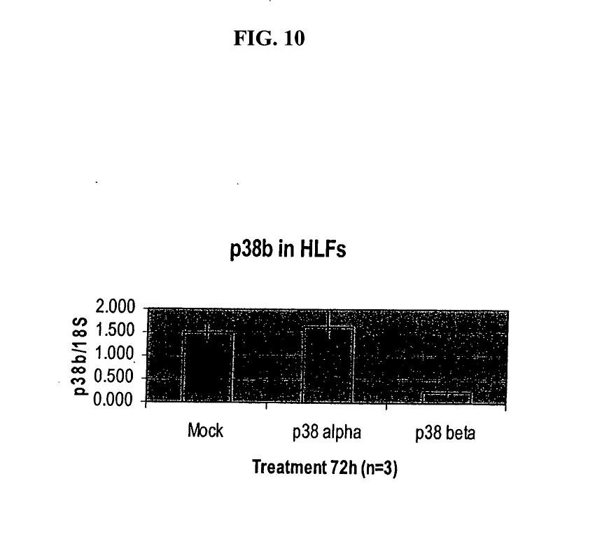

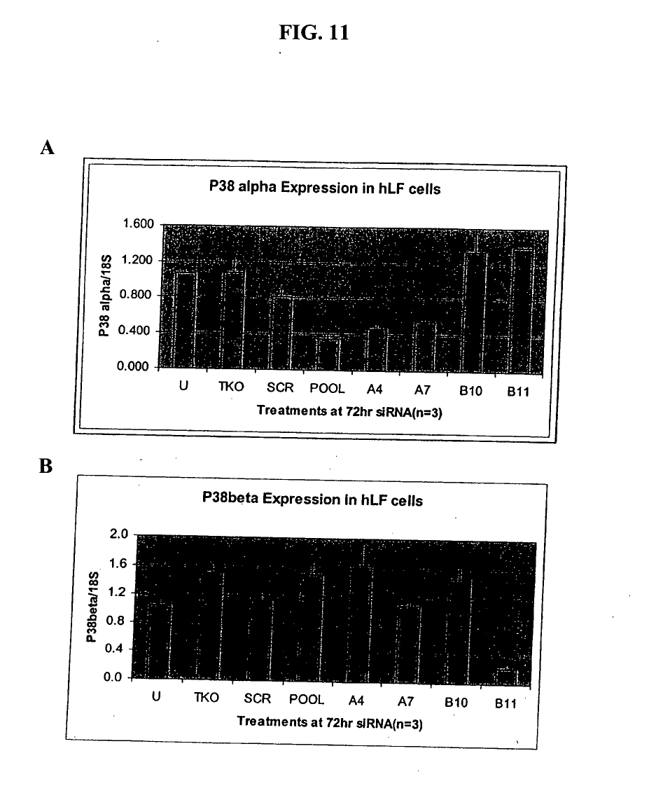

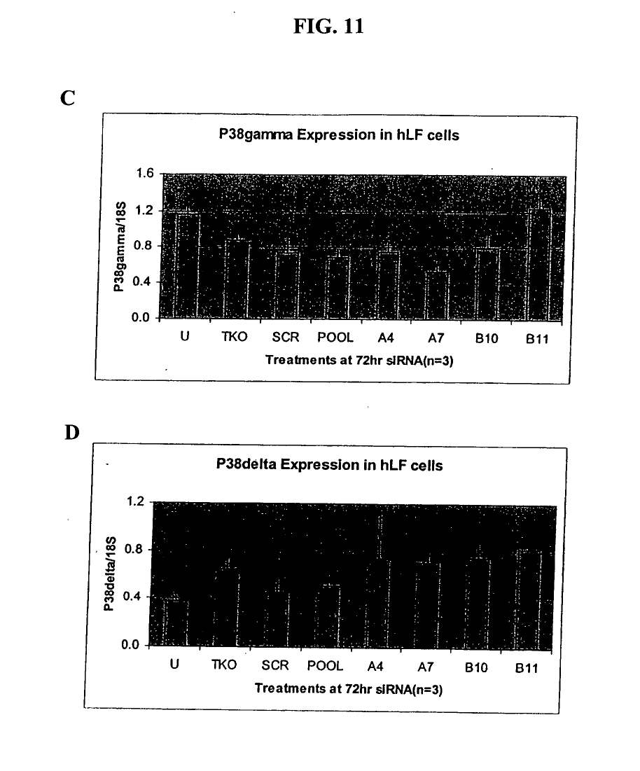

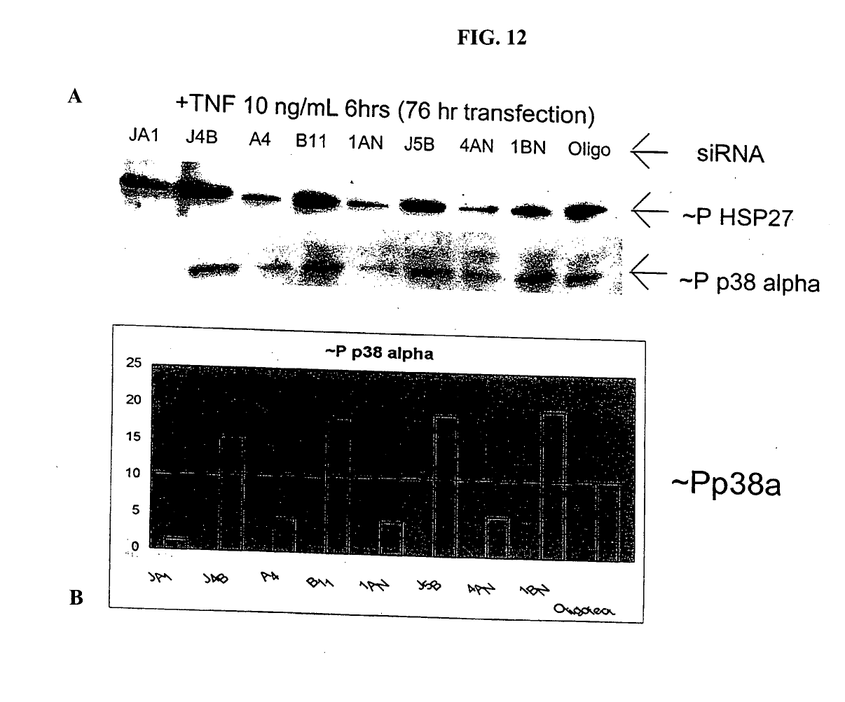

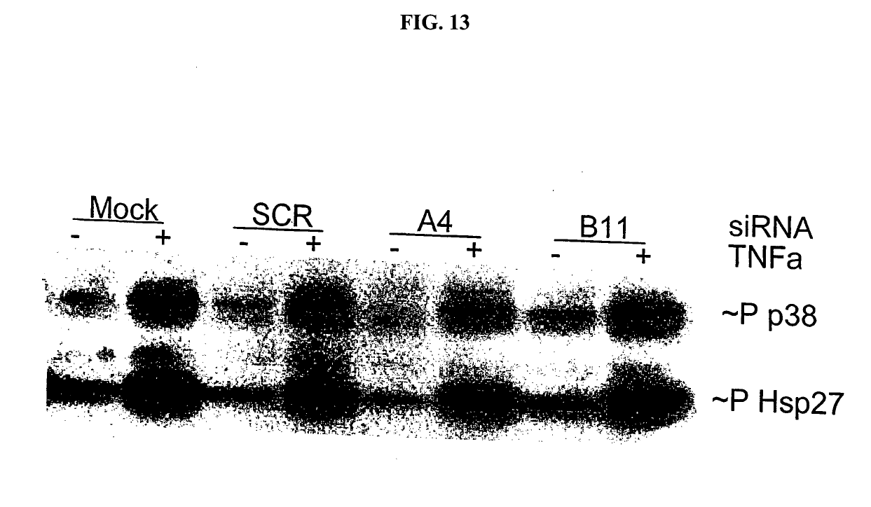

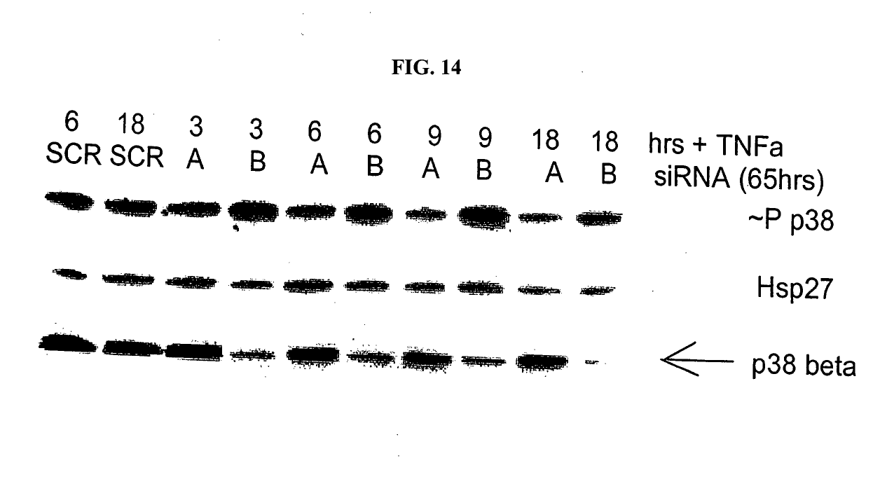

The impact of p38 MAP kinase-directed siRNAs was examined with respect to another downstream marker of the p38 MAP kinase pathway, interleukin 1-B. IL-1B is known to play a role in the inflammatory response. As shown in FIG. 10, cells treated with the p38-α-directed siRNA A4 showed comparable levels of IL-1B RNA as cells treated with a mock transfection or a control siRNA (SCR). In contrast, cells transfected with the p38β-directed siRNA B11 showed an increase in IL-1B RNA. These results further support the observation that inhibition of p38β kinase leads to a compensatory response by other p38 MAP kinases, which in turn, leads to downstream events in the p38 MAP kinase pathway.

Finding: Knock down of p38-β expression caused by p38-β-directed siRNA, confirmed by Western analysis showing loss of p38-β protein, paradoxically results in increase in total ˜P-p38. Preliminary results suggest that the increase in downstream phosphorylation events is caused by an increase in mRNA levels for the α, γ, and δ isoforms of the p38 MAP kinase. That the increased ˜P-p38 results in increased activity, as would be expected, is confirmed by demonstration of increased phosphorylation of the downstream substrate Hsp27 and of increased gene expression for members of the p38α activation cassette COX-2, IL-1, and possibly IL-6.

siRNA Sequences

| | | |
|---|---|---|
| JA1) | | |
| Sense Sequence: | UCUCCGAGGUCUAAAGUAUdAdT; | (SEQ ID NO: 1) |
| Antisense Sequence: | AUACUUUAGACCUCGGAGAdAdT; | (SEQ ID NO: 2) |
| J4B) | | |
| Sense Sequence: | GCACGUUCAAUUCCUGGUUdTdA; | (SEQ ID NO: 3) |
| Antisense Sequence: | AACCAGGAAUUGAACGUGCdTdC; | (SEQ ID NO: 4) |
| A4) | | |
| Sense Sequence: | CCUACAGAGAACUGCGGUUdTdT; | (SEQ ID NO: 5) |
| Antisense Sequence: | AACCGCAGUUCUCUGUAGGdTdT; | (SEQ ID NO: 6) |
| B11) | | |
| Sense Sequence: | GGACCUGAGCAGCAUCUUCdTdT; | (SEQ ID NO: 7) |
| Antisense Sequence: | GAAGAUGCUGCUCAGGUCCdTdT; | (SEQ ID NO: 8) |
| 1AN) | | |
| Sense Sequence: | GUCCAUCAUUCAUGCGAAAdTdT; | (SEQ ID NO: 9) |
| Antisense Sequence: | UUUCGCAUGAAUGAUGGACdTdT; | (SEQ ID NO: 10) |
| J5B) | | |
| Sense Sequence: | GGAGCUCACUUACCAGGAAdGdT | (SEQ ID NO: 11) |
| Antisense Sequence: | UUCCUGGUAAGUGAGCUCCdTdT | (SEQ ID NO: 12) |
| 4AN) | | |
| Sense Sequence: | GAAGCUCUCCAGACCAUUUdTdT | (SEQ ID NO: 13) |
| Antisense Sequence: | AAAUGGUCUGGAGAGCUUCdTdT | (SEQ ID NO: 14) |
| 1BN) | | |
| Sense Sequence: | GCGACUACAUUGACCAGCUdTdT | (SEQ ID NO: 15) |
| Antisense Sequence: | AGCUGGUCAAUGUAGUCGCdTdT | (SEQ ID NO: 16) |

### EXAMPLE 2

### Inhibition of the p38 α-Isoform by siRNA and by Direct Inhibition Reduces Levels of Phosphorylated Downstream Substrate Hsp27

Using the methods described in Example 1, a Western blot was prepared using untreated cells, cells containing a control siRNA (SCR), and cells containing either a p38-α-directed siRNA (A) or a p38-β-directed siRNA (B). The Western blot compares cells were either treated or untreated with 10 ng of the inflammatory cytokine TNF-α. One lane shows cells that were untreated with siRNA, but that received both TNF-α and an inhibitor of p38 MAP kinase that is approximately 200-fold more selective for the α-isoform. All cells exposed to the inflammatory cytokine TNFα showed some level of Hsp27 phosphorylation, except the untreated cells that received both the cytokine and the selective p38 MAP kinase inhibitor. These results indicate that the inhibitor is effective at blocking the inflammatory signal provided by TNF-α.

Cells that received both the TNFα stimulant as well as the p38α-directed siRNA showed a reduced level of Hsp27 phosphorylation as compared to the untreated cells. These results indicate that the p38α-directed siRNA is effective at substantially blocking or at least attenuating the inflammatory signal provided by TNF-α. In contrast, cells that received both the TNF-α stimulant as well as the p38β-directed siRNA showed a level of Hsp27 phosphorylation that was higher than the SCR control cells and comparable to the untreated cells exposed to the inflammatory cytokine. These results indicate that cells that received either a selective inhibitor of p38α kinase or a p38α-directed siRNA were less sensitive to the impact of the inflammatory cytokine TNF-α, while cells that received a p38-β-directed siRNA remained sensitive to the cytokine.

The data from this experiment suggest that inhibitors of p38 MAP kinase that are not selective for the alpha isoform may be ineffective in blocking the signal cascade of the p38 MAP kinase pathway. Such non-selective inhibitors would be less likely to be effective as treatments for inflammation and other disease states associated with an activated p38 MAP kinase pathway.

### EXAMPLE 3

### Higher Concentration of p38 MAP Kinase Isoform Non-Specific Inhibitor Diminish Potency

Mitogen-activated protein kinase p38 (p38) is activated (phosphorylated) by stress and proinflammatory signals. p38 MAP kinase activates phospholipase A2 (PLA2). PLA2 liberates arachidonic acid that cyclooxygenase (COX) converts to prostaglandins such as PGE2. Thus, PGE2 production is an indirect marker for p38 MAP kinase activity.

The impact of increasing doses of a p38 MAP kinase inhibitor that was approximately 10× fold more specific for the α-isoform of the enzyme was studied using an Escherichia coli lipopolysaccharide (LPS) inflammation model. As discussed below, high doses of the inhibitor began to reverse or disinhibit the inhibitory function of the compound. These results support observation that inhibition of the β-isoform of the p38 MAP kinase results in the upregulation of other components of the p38 MAP kinase pathway.

To determine the impact of higher concentrations of a non-specific p38 inhibitor, venous blood was collected from healthy male beagle dogs (SRI, Palo Alto, Calif.) into heparinized syringes and used within 2 hours of collection. The p38 inhibitor Compound 5 the Exemplary inhibitors was dissolved in 100% DMSO and 1 ml aliquots of drug concentrations ranging from 0 to 20 mM (final drug concentrations in incubation mixture equal 0 to 20 mM) were dispensed into triplicate wells of a 24-well microtiter plate (NUNCLON DELTA SI, Applied Scientific, So. San Francisco, Calif.).

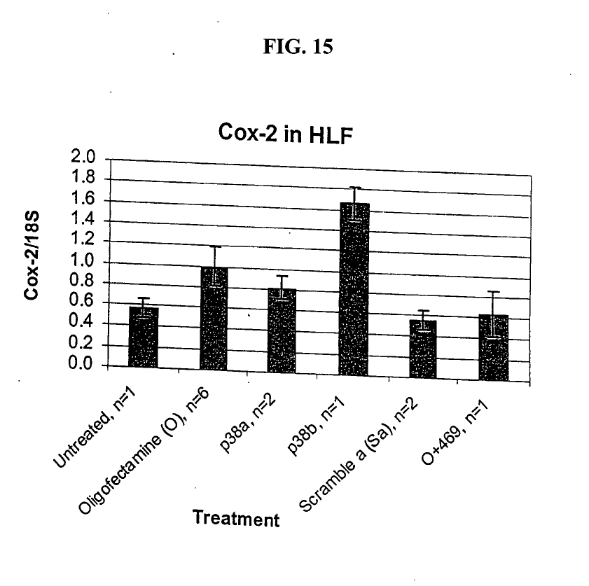

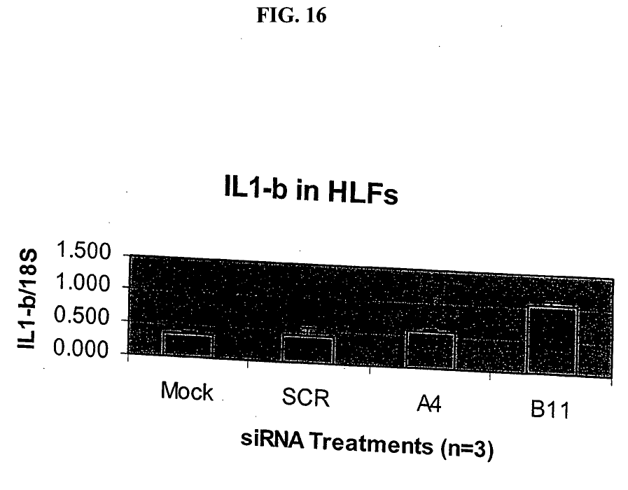

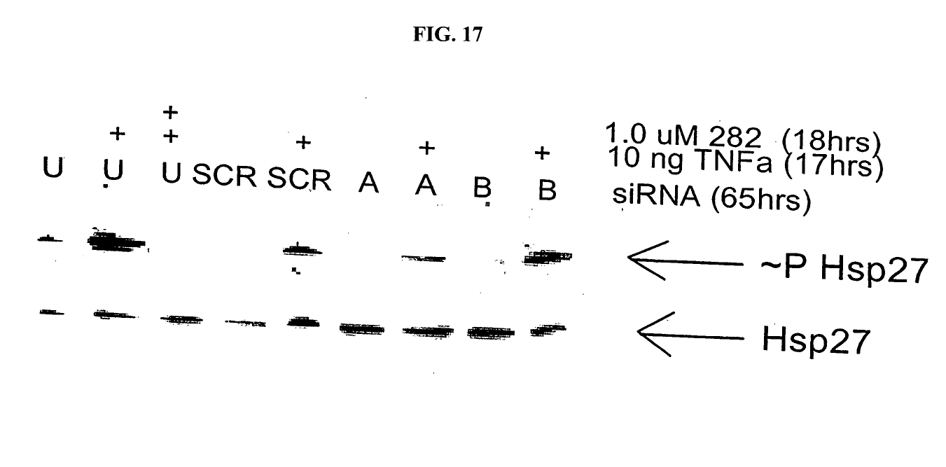

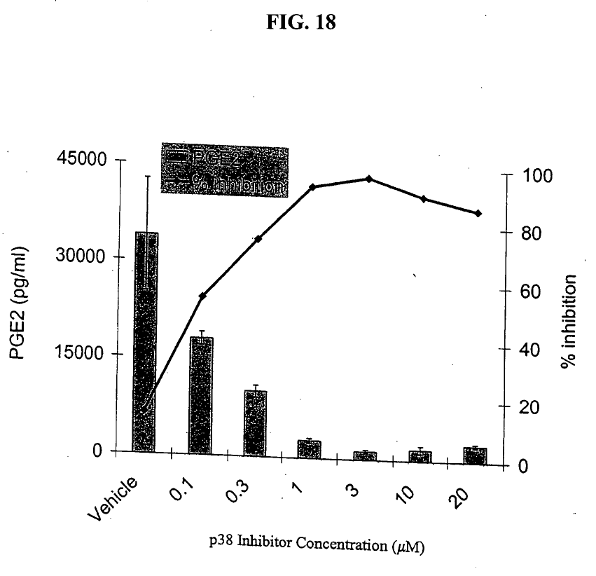

Whole blood was added at a volume of 1 ml/well and the mixture incubated for 15 minutes with constant shaking (TITER PLATE SHAKER, Lab-Line Instruments, Inc., Melrose Park, Ill.) at a humidified atmosphere of 5% CO₂ at 37° C. Whole blood was cultured undiluted. At the end of the initial incubation period, 10 ml of LPS (E. coli 0111:B4, Sigma Chemical Co., St. Louis, Mo.) was added to each well to a final concentration of 10 mg/ml. The incubation was continued for an additional 24 hours under the same initial conditions. The reaction was stopped by placing the microtiter plates in an ice bath and plasma or cell-free supernates were collected by centrifugation at 3000 rpm for 10 minutes at 4° C. The plasma samples were stored at −80° C. until assayed for PGE₂ levels by ELISA, following the directions supplied by High Sensitivity Colorimetric Competitive ELISA PGE2 assay kit (R&D Systems, Minneapolis, Minn.). The results from the experiment were expressed as % PGE2 inhibition and are shown in FIG. 15.

The “No Trtmt” (no treatment) and “Vehicle” columns indicate the background level of PGE2 production in the assay system. The impact of increasing concentrations of the p38 inhibitor Compound 5 is shown alone the X-axis of the graph. The highest level of inhibition was observed when the treated material was subjected to a 3 μM concentration of the inhibitor. Interestingly, disinhibition of PGE2 production was observed at higher concentrations of the inhibitor (10 μM and 20 μM).

These results support the observations regarding the present invention which provide that inhibition of the β-isoform of p38 MAP kinase causes an upregulation of other elements of the p38 MAP kinase pathway, which in turn results in an upregulation of those systems regulated by p38 MAP kinase. In the present experiment, when the p38 MAP kinase inhibitor Compound 5 is provided to the system, the relative non-selective 10-fold preference of the inhibitor for the alpha-isoform over the beta-isoform is sufficient to inhibit the system and thus reduce the about of PGE2 produced. However, as the concentration of the inhibitor increases, more of it interacts with and inhibits the activity of the beta-isoform of the kinase. As observed with the siRNA experiments discussed above, inhibition of p38-β MAP kinase results in an increase in downstream events of the p38 MAP kinase pathway. This effect is seen here with the disinhibition of PGE2 production in the presence of the p38 inhibitor Compound 5 at concentrations of 10 μM and higher. Thus, the potency of this p38 inhibitor decreases with increasing concentrations of the relatively non-isoform specific inhibitor.

Although the invention has been described with reference to embodiments and examples, it should be understood that various modifications can be made without departing from the spirit of the invention. Accordingly, the invention is limited only by the following claims. All references cited herein are hereby expressly incorporated by reference.

1. A method for designing an isoform selective p38-α MAP kinase inhibitor, comprising:
providing a model of an ATP bind site of a p38-α MAP kinase;
providing a candidate compound capable of binding to the ATP binding site;
designing the isoform selective inhibitor by selecting one or more substituents for the candidate compound that mediates the isoform selective inhibition of p38-α MAP kinase, wherein the inhibitor is at least 150 fold more selective for p38-α than for p38-β, and wherein the inhibitor is more potent than an p38 MAP kinase isoform non-specific inhibitor.

2. The method of claim 1, wherein the designing step further comprises selecting one or more substituents that mediate selective inhibition of p38-α MAP kinase which are a hydrophobic substituent or a hydrogen bond acceptor substituent.

3. The method of claim 2, wherein the hydrophobic substituent is selected from the group consisting of an aryl group, a substituted aryl group, a lower alkyl group, a substituted lower alkyl, and a halo group.

4. The method of claim 2, wherein the hydrogen bond acceptor substituent is a carbonyl group.

5. The method of claim 1, wherein the designing step further comprises selecting one or more substituents to form a first hydrophobic region, a hydrogen bond acceptor region and a second hydrophobic region.

6. The method of claim 5, wherein the designing step further comprises locating the first hydrophobic region approximately 6.8±3 Å from the hydrogen bond acceptor region.

7. The method of claim 5, wherein the designing step further comprises locating the second hydrophobic region approximately 3.9±2.5 Å from the hydrogen bond acceptor region.

8. The method of claim 5, wherein the designing step further comprises locating the first hydrophobic region approximately 9.2±2.5 Å from the second hydrophobic region.

9. A pharmacophore comprising:
a core;
a first hydrophobic region attached to the core;
a hydrogen bond acceptor region attached to the core; and
a second hydrophobic region attached to the core, wherein the hydrogen bond acceptor region and the second hydrophobic region are positioned to facilitate isoform independent binding to an ATP binding site of a p38 MAP kinase, and wherein the second hydrophobic region is positioned to convey selective binding of the pharmacophore to the ATP bind site of p38-α MAP kinase such that the inhibitor is at least 150 fold more selective for p38-α than for p38-β, and wherein the inhibitor is more potent than an isoform non-specific inhibitor.

10. The pharmacophore of claim 9, wherein the first hydrophobic region, the hydrogen bond acceptor region and the second hydrophobic region for a plane.

11. The pharmacophore of claim 9, wherein the first hydrophobic region is approximately 6.8±3 Å from the hydrogen bond acceptor region.

12. The pharmacophore of claim 9, wherein the second hydrophobic region approximately 3.9±2.5 Å from the hydrogen bond acceptor region.

13. The pharmacophore of claim 9, wherein the first hydrophobic region approximately 9.2±2.5 Å from the second hydrophobic region.

14. The pharmacophore of claim 9, wherein the hydrogen bond acceptor region forms a hydrogen bond with an amide proton of MET109 of p38-α MAP kinase.

15. The pharmacophore of claim 9, wherein the second hydrophobic region forms a hydrophobic interaction with ALA40 of p38-α MAP kinase.

16. The pharmacophore of claim 15, wherein the pharmacophore is at least 150 fold more selective for p38-α MAP kinase than for p38-β MAP kinase.

17. A method of treating a condition caused or exacerbated by p38 MAP kinase activity comprising:
identifying a subject suffering from the condition caused or exacerbated by p38 MAP kinase activity; and
administering a potent p38 MAP kinase isoform specific inhibitor to the subject, wherein the inhibitor binds to an ATP binding site of a p38 MAP kinase and wherein the inhibitor is at least 150 fold more selective for p38-α than for p38-β, whereby the inhibitor is more potent than an isoform non-specific inhibitor.

18. The method of claim 17, wherein the p38 MAP kinase inhibitor is a pharmacophore comprising a core with a first hydrophobic region, a hydrogen bond acceptor region, and a second hydrophobic region, wherein the hydrogen bond acceptor region and the second hydrophobic region are positioned to facilitate isoform independent binding to an ATP binding site of a p38 MAP kinase, and wherein the second hydrophobic region is positioned to convey selective binding of the pharmacophore to the ATP bind site of p38-α MAP kinase.

19. The method of claim 18, wherein the second hydrophobic region forms a hydrophobic interaction with ALA40 of p38-α MAP kinase.

20. The method of claim 19, wherein the hydrogen bond acceptor region forms a hydrogen bond with an amide proton of MET109 of p38-α MAP kinase
